(19) United States
(12) Patent Application Publication
(10) Pub. No.: US 2009/0165220 A1
(43) Pub. Date: Jul. 2, 2009

(54) Depilatory products

(76) Inventors: Leonard Mackles, New York, NY (US); William Bass, Edison, NJ (US)

Correspondence Address: OMRI M. BEHR, 325 PIERSON AVENUE, EDISON, NJ, 08837-3123, US

(21) Appl. No.: 12/315,566

(22) Filed: Dec. 4, 2008

Related U.S. Application Data
(60) Provisional application No. 61/016,638, filed on Dec. 26, 2007.

(51) Int. Cl. A61K 8/22; A61Q 9/04

(52) U.S. Cl. 8/161

(57) ABSTRACT

There is provided a shelf stable two part aqueous based depilatory system comprising an acidic oxidative phase and a basic reductive phase containing, inter alia at least one depilatory compound, wherein the mixing of the two phases provides a substantially neutral warm depilatory mixture.

### RELATED APPLICATIONS

This application claims priority of copending provisional application 61/016,638 filed Dec. 26, 2007

### FIELD OF THE INVENTION

Self warming aqueous depilatory products which are effective on the mixing of their components. The combination of the oxidation-reduction and acid-base reactions of the component phases produce heat as well as depilatory action.

### BACKGROUND OF THE INVENTION

Depilatory products have been used for many years to remove unwanted hair from the human body. At present there are numerous depilatory products available commercially, the vast majority of which utilize a thioglycolic acid salt at alkaline pH as the depilatory agent. One means of increasing the effectiveness as well as the comfort of using these products is to incorporate some type of warming technology. The warm temperature increases the rate at which the depilatory will react with the hair, thus shortening the time required to remove the hair, as well as, making the application more cosmetically appealing to the user.

### DISCUSSION OF THE PRIOR ART

The use oxidation-reduction reactions is a well known art for producing warm topical products. For example, U.S. Pat. No. 7,008,620 describes one application of this technology for use on a dry substrate.

### SUMMARY OF THE INVENTION

The present invention deals with an aqueous based, two-phase emulsion system, which includes a stabilizer for the oxidation phase. In addition to stabilizing the oxidizing agent, the stabilizer adds to the heat produced when the two phases are mixed.

In the present invention, a dermally acceptable peroxide, suitably, hydrogen peroxide, is used as the oxidizing agent in an emulsion vehicle containing one or more emulsifiable organic compounds and emulsion forming agent(s). As is well known, peroxides will readily oxidize most organic compounds. In order to insure adequate shelf-life stability of the product, an inorganic acid, suitably a strong acid, stabilizer is added to the oxidizing emulsion to inhibit the oxidation of the organic compounds by the peroxide. The addition of the inorganic acid causes the pH of the oxidizing phase to be acidic. When the oxidizing phase is combined with the reducing agent phase, which is at alkaline pH, the resulting reaction between the inorganic acid, and alkaline pH of the reducing phase results in an increase in the amount of heat generated over that which would have been produced if the inorganic acid was not present. Thus:

Reducing Phase+Oxidizing Phase (w/o acid) gives a +40° F. temperature rise.

Reducing Phase+Oxidizing Phase (with Strong Acid) gives a +60° F. temperature rise.

The reducing agent phase contains an excess of the depilatory agent over the oxidizing phase. A portion of the depilatory agent acts as the reducing agent in the oxidation-reducing reaction. (as in U.S. Pat. No. 7,008,620) the remainder acts as the depilatory.

### DESCRIPTION OF THE PREFERRED EMBODIMENTS

All quantities herein are w/w % based on the total composition of the phase to which they refer. The core of the oxidation phase comprises a cosmetically acceptable emulsifiable organic compound, such as mineral oil or a fatty alcohol, suitably a C₈₋₂₀ alcohol, preferably a C_{10 -18} alcohol, at about 1-10%, preferably about 1-5%, emulsifiers 1-20% suitably about 1-5% and hydrogen peroxide (suitably 50% aq.) about 5-20% (measured as a 50% solution).

It is well known that hydrogen peroxide will react with organic compounds which can result in instability of the emulsion, a reduction in peroxide content, and off-odors being produced. In order to prevent this, in the preferred phase of this invention, strong inorganic acids are added which would include phosphoric, sulfuric, hydrochloric, nitric, with phosphoric preferred. The amount of acid (based on aqueous acid, is suitably between about 0.025-5% preferably between about 0.05-1%). Acid acts as a stabilizer for the emulsion. A secondary benefit is discussed below. The remaining component is water.

The reducing phase comprises the depilatory agents suitably thioglycolic acid salts of alkali and alkali earth metals. The total thioglycolate, measured as thioglycolic acid is between about 5 and 30% provided that this is an excess over the amount required to completely react with the peroxide, suitably a 5-15%, preferably a 5-10% excess over peroxide. The phase also contains a cosmetically acceptable emulsifiable organic compound, such as mineral oil or a fatty alcohol, suitably a C₈₋₂₀ alcohol, preferably a C₁₀₋₁₈ alcohol, at about 1-10%, preferably about 1-5%, emulsifiers about 1-20% suitably about 1-5% and a base suitably an alkali or alkaline earth metal base, suitably the hydroxide, of suitably sodium, potassium, magnesium or calcium, preferably calcium, of between about 5 and 15%.

In the core reaction of formula Example 1, the hydrogen peroxide reacts with a portion of the calcium and potassium thioglycolates to produce heat. This oxidation/reduction reaction is discussed in U.S. Pat. No. 7,008,620. The thioglycolates are reducing agents which are also depilatory agents. During the reaction, the portions of the thioglycolates are oxidized to compounds which would not have any depilatory activity. Hence an excess of thioglycolates is required so that the un-reacted thioglycolates can produce the depilatory action.

The inorganic acid has a second effect on the system when the two phases are mixed. The inorganic acid reacts with some of the base, suitably calcium hydroxide that has been formulated into the reducing phase. When the inorganic acid reacts with the calcium hydroxide it produces additional heat above and beyond that which is produced by the oxidation-reduction reaction. As used and claimed herein the w/w % given refers to the aqueous form used, not the non aqueous form.

### EXAMPLE

### Formula Example 1

| | |
|---|---|
| Ingredient | % |

Oxidation phase (w/o acid)

| | |
|---|---|
| Water | 89.50 |
| Cetyl Alcohol | 2.50 |
| Glyceryl Monostearate | 2.50 |
| Hydrogen Peroxide (50%) | 5.50 |
| Total | 100.00 |

Reducing Phase

| | |
|---|---|
| Water | 38.00 |
| Calcium Thioglycolate | 14.00 |
| Potassium Thioglycolate (43%) | 30.00 |
| Phosphate Ester 785 | 4.00 |
| Sorbitan Monooleate | 5.00 |
| Cetyl Alcohol | 4.00 |
| Calcium Hydroxide | 5.00 |
| Total | 100.00 |

The oxidizing phase of the above formulation has an insufficient level of shelf stability for this example to be commercially viable.

### Formula Example 2

| | |
|---|---|
| Ingredient | % |

Oxidation Phase (w acid)

| | |
|---|---|
| Water | 84.50 |
| Cetyl Alcohol | 2.50 |
| Glyceryl Monostearate | 2.50 |
| Hydrogen Peroxide (50%) | 5.50 |
| Phosphoric Acid (38%) | 5.00 |
| Total | 100.00 |

Reducing Phase

| | |
|---|---|
| Water | 35.00 |
| Calcium Thioglycolate | 14.00 |
| Potassium Thioglycolate (43%) | 30.00 |
| Phosphate Ester 785 | 4.00 |
| Sorbitan Monooleate | 5.00 |
| Cetyl Alcohol | 4.00 |
| Calcium Hydroxide | 10.00 |
| Total | 100.00 |

### Additional Formula Examples

| | | | |
|---|---|---|---|
| 3 | 4 | 5 | 6 |

| | |
|---|---|
| Ingredient | % |

Oxidation Phase (w acid)

| | | | | |
|---|---|---|---|---|
| Water | 89.00 | 86.25 | 85.00 | 78.50 |
| Cetyl Alcohol | 2.50 | — | 4.00 | — |
| Glyceryl Monostearate | 2.50 | — | — | — |
| Sorbitan Tristearate | — | 3.00 | 1.00 | 5.00 |
| Tween 20 | — | 1.75 | 3.00 | 2.50 |
| Mineral Oil | — | 2.00 | — | 5.00 |
| Hydrogen Peroxide (50%) | 5.50 | 4.00 | 6.00 | 5.50 |
| Phosphoric Acid (38%) | — | 3.00 | 1.00 | 3.50 |
| Hydrochloric Acid (50%) | 0.50 | — | — | — |
| Total | 100.00 | 100.00 | 100.00 | 100.00 |

Reducing Phase

| | | | | |
|---|---|---|---|---|
| Water | 35.00 | 47.00 | 31.50 | 37.50 |
| Calcium Thioglycolate | 14.00 | 14.00 | — | 14.00 |
| K Thioglycolate (43%) | 30.00 | 17.50 | 51.00 | 30.00 |
| Phosphate Ester 785 | 4.00 | — | 4.00 | 1.00 |
| Sorbitan Monostearate | — | 5.00 | 5.00 | 3.00 |
| PEG-40 Stearate | — | 3.50 | — | 2.50 |
| Sorbitan Monooleate | 5.00 | — | — | — |
| Cetyl Alcohol | 4.00 | 5.00 | 2.50 | 3.50 |
| Calcium Hydroxide | 8.00 | 8.00 | 6.00 | 8.50 |
| Total | 100.00 | 100.00 | 100.00 | 100.00 |

1. A method of hair removal from a mammal comprising the sequential steps of mixing
a) an oxidation phase consisting essentially of a cosmetically acceptable emulsifiable organic compound, emulsifying agent, dermally acceptable peroxide, inorganic acid and water and
b) a reducing phase consisting essentially of thioglycolate, a cosmetically acceptable emulsifiable organic compound, emulsifying agent, and base
wherein the amount of peroxide is insufficient to oxidize all the thioglycolate and the acid is insufficient to neutralize all of the base, to provide a warm depilatory mixture having a temperature above the initial temperatures of the components, thereafter
applying said warm depilatory phase to the hair which it is desired to remove.

2. The method of claim 1 wherein cosmetically acceptable emulsifiable organic compound is mineral oil or a fatty alcohol.

3. The method of claim 1 wherein the acid is strong inorganic acid.

4. The method of claim 3 wherein the acid is selected from the group consisting of sulfuric, nitric, hydrochloric and phosphoric acids.

5. The method of claim 4 wherein the acid is at least one member selected from the group consisting of hydrochloric and phosphoric acids.

6. The method of claim 1 wherein the oxidation phase, as w/w % based on total content, consists essentially of:
C₁₀₋₁₈ alcohol about 1-10%, hydrogen peroxide about 5-20%, strong acid about 0.025 to 5.0% emulsifiers about 1-20%, and water to 100% and
wherein the reduction phase, as w/w % based on total content, consists essentially of: total thioglycolate measured as thioglycolic acid about 5-30%, C₁₀₋₁₈ alcohol about 1-10%, emulsifiers about 1-20%, alkali metal base about 1-5% and water to 100%, provided that said total glycolate is an excess of 5-15 mol % of said hydrogen peroxide.

7. The method of claim 6 wherein C₁₀₋₁₈ alcohol is between about 1-5% acid is between about 0.05 and 1% and emulsifiers are between about 1-5%, in each phase.

8. The method of claim 7 wherein the acid is phosphoric acid, the base is calcium hydroxide, and thioglycolate is at least one member of the group consisting of calcium and potassium thioglycolates

9. A kit comprising first and second containers,
said first container charged with an oxidation phase consisting essentially of a cosmetically acceptable emulsifiable organic compound, an emulsifying agent, a dermally acceptable peroxide, an inorganic acid and water and
a second container charged with a reducing phase consisting essentially of a thioglycolate, cosmetically acceptable emulsifiable organic compound, an emulsifying agent, and a base
wherein the amount of peroxide is insufficient to oxidize all the thioglycolate and the acid is insufficient to neutralize all of the base,
wherein the mixing of the contents of the containers provides a warm depilatory mixture having a temperature above the initial temperatures of the components.

10. The kit of claim 9 wherein cosmetically acceptable emulsifiable organic compound is mineral oil or a fatty alcohol.

11. The kit of claim 9 wherein the acid is strong inorganic acid.

12. The kit of claim 11 wherein acid is selected from the group consisting of sulfuric, nitric, hydrochloric and phosphoric acids.

13. The kit of claim 12 wherein acid is at least one member selected from the group consisting of hydrochloric and phosphoric acids.

14. The kit of claim 9 wherein the oxidation phase, as w/w % based on total content, consists essentially of:
C₁₀₋₁₈ alcohol about 1-10%, hydrogen peroxide about 5-20%, strong acid about 0.025 to 5.0% emulsifiers about 1-20%, and water to 100% and
wherein the reduction phase, as w/w % based on total content, consists essentially of: total thioglycolate measured as thioglycolic acid about 5-30%, C₁₀₋₁₈ alcohol about 1-10%, emulsifiers about 1-20%, alkali metal base about 1-5% and water to 100%, provided that said total glycolate is an excess of 5-15 mol % of said hydrogen peroxide.

15. The kit of claim 14 wherein the C₁₀₋₁₈ alcohol is between about 1-5%, acid is between about 0.05 and 1% and the emulsifiers are between about 1-5%, in each phase.

16. The kit of claim 13 wherein the acid is phosphoric acid, the base is calcium hydroxide, and the thioglycolate is at least one member of the group consisting of calcium and potassium thioglycolates.
